(19) United States
(12) Patent Application Publication
(10) Pub. No.: US 2024/0024868 A1
(43) Pub. Date: Jan. 25, 2024

(54) MICROFLUIDIC DETECTION DEVICE

(71) Applicant: National Central University, Taoyuan City (TW)

(72) Inventors: Ching-Yun Chen, Taoyuan City (TW); Cherng-Jyh Ke, Taoyuan City (TW); Shih-Tien Hsu, Taichung City (TW); Ching-Wen Tsai, Hsinchu City (TW); Si-Ting Wu, Kaohsiung City (TW); Yi-Hsuan Tung, Taichung City (TW); Hsin-I Chiu, Pingtung County (TW); Sheng-Wen Chang, Hsinchu City (TW); Jing-Ke Chen, Hsinchu County (TW); Yueh-Teng Tsai, New Taipei City (TW); Ching Yu, Kaohsiung City (TW); Jian-Hua Chang, Taipei City (TW)

(21) Appl. No.: 18/226,216

(22) Filed: Jul. 25, 2023

Related U.S. Application Data
(60) Provisional application No. 63/369,341, filed on Jul. 25, 2022.

(51) Int. Cl. B01L 3/00

(52) U.S. Cl. CPC B01L 3/502715; B01L 3/5025; B01L 2400/0633; B01L 2300/0816; B01L 2200/16; B01L 2300/087; B01L 2300/0864

(57) ABSTRACT

Provided is a microfluidic detection device, including a base with a microfluidic channel structure formed on the base and a lid covering the base. The microfluidic channel structure includes a sample well for loading a sample, a detection well having a first reagent for reacting with the sample, and a channel connecting the sample well and the detection well. The detection well has a recess deeper than the channel, and the base includes a protrusion corresponding to the recess to form a space between the protrusion and the recess. Also provided is a method for rapid diagnostic testing by the microfluidic detection device.

### CROSS REFERENCE TO RELATED APPLICATIONS

This application claims the priority to and benefit of U.S. Provisional Application No. 63/369,341, filed on Jul. 25, 2022. The entire content thereof is incorporated herein by reference.

### BACKGROUND OF THE INVENTION

### 1. Field of the Invention

The present disclosure relates to a microfluidic detection device, and particularly to a microfluidic detection device for rapid diagnostic testing (RDT).

### 2. Description of the Prior Art

Cervical cancer is one of the deadliest cancers around the globe among women according to the report of the World Health Organization (WHO), and more than 90% are caused by human papillomavirus (HPV) infection. HPV can cause a variety of diseases and is divided into different types according to the cancer risk. Among these, HPV-16 is a high-risk virus type that causes cervical cancer primarily. In addition to cervical cancer, HPV-16 is also the main type of virus that causes various severe cancers such as head and neck cancers.

Recently, it is not difficult to detect biochemical values and disease pathogens from samples through immunoassay. There are many rapid diagnostic tests (RDTs) available on the market, including RDTs for Covid-19, ovulation (luteinizing hormone, LH), Influenza A/B, and pregnancy (human chorionic gonadotropin, hCG).

However, for some specific diseases, such as cervical cancer and other cancer, there are currently no RDT products for immunoassays. One reason is the inherently low levels of disease pathogens or target molecules in the human body or samples. The color markers, e.g. antibody-colloidal gold conjugates, contained in RDTs bind to the target molecules in the samples through immunoreaction and are captured in the test line to develop color, but too little target molecules results in poor color development on RDTs. Hence, the RDT for specific diseases, especially cervical cancer, are not applicable.

Accordingly, there is an urgent and unmet need in the art to provide a microfluidic detection device that can solve the problems mentioned above.

### SUMMARY OF THE INVENTION

To solve the aforementioned problems, the present disclosure provides a microfluidic detection device, comprising: a base with a microfluidic channel structure formed on the base, wherein the microfluidic channel structure comprises: a sample well for loading a sample, a detection well has a first reagent for reacting with the sample, and a channel connecting the sample well and the detection well, wherein the detection well has a recess deeper than the channel; and a lid covering the base, having: a protrusion corresponding to the recess to form a space between the protrusion and the recess.

In at least one embodiment of the present disclosure, the recess is along the direction from an upper surface of the base to a lower surface of the base.

In at least one embodiment of the present disclosure, the recess and the protrusion are arc-shaped. In some embodiment, the space between the recess and the protrusion forms a curved channel.

In at least one embodiment of the present disclosure, the first reagent comprises a signal booster.

In at least one embodiment of the present disclosure, the lid further has at least one air hole corresponding to the microfluidic channel structure.

In at least one embodiment of the present disclosure, the detection well has a control region and a test region.

In at least one embodiment of the present disclosure, the lid further has: a sample through hole corresponding to the sample well; and a detection window corresponding to the detection well.

In at least one embodiment of the present disclosure, the microfluidic channel structure further comprises a reaction well between the sample well and the detection well, the reaction well has a second reagent for reacting with the sample, and wherein the sample well, the reacting well, and the detection well are connected by the channel. In some embodiment, the reaction well is deeper than the channel along the direction from the upper surface of the base to the lower surface of the base. In some embodiment, the microfluidic channel structure is branched, and each branch of the microfluidic channel structure comprises the reaction well and the detection well. In some embodiment, the microfluidic detection device further comprises a gate disposed between the reaction well and the detection well to control the flow of fluid from the reaction well to the detection well. In some embodiment, the second reagent comprises antibody-colloidal gold conjugates. In some embodiment, the antibody-colloidal gold conjugates are coated on a test paper disposed on the reaction well. In some embodiment, the second reagent comprises a reagent for nucleic acid amplification. In some embodiment, the nucleic acid amplification is selected from the group consisting of nucleic acid sequence-based amplification (NASBA), recombinase polymerase amplification (RPA), loop-mediated isothermal amplification (LAMP), strand displacement amplification (SDA), helicase-dependent amplification (HDA), rolling circle amplification (RCA), and whole genome amplification (WGA).

In at least one embodiment of the present disclosure, the present disclosure further provides a method for rapid diagnostic testing, comprising: obtaining a sample from a subject in need thereof; loading the sample to the microfluidic detection device of the present disclosure; and detecting a biochemical value or a disease pathogen by the microfluidic detection device. In some embodiment, the disease pathogen is selected from the group consisting of bacteria, viruses, fungi, protists, and parasitic worms.

In conclusion, the microfluidic detection device of the present disclosure has a recess set between a control region and a test region on a base, and a protrusion set on a lid corresponding to the recess that enable the sample flowing through the test region longer, and the impurities and unwanted substances deposited at the bottom of the recess, thereby improving the color development. Moreover, the preset disclosure has a signal booster provided in the space between the recess and the protrusion to further enhance the specificity of the target molecules from the sample.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram illustrating a microfluidic detection device according to an embodiment of the present invention.

FIG. 2 is a schematic diagram illustrating a microfluidic detection device according to an embodiment of the present invention.

FIG. 3 is a schematic diagram illustrating a microfluidic detection device according to an embodiment of the present invention.

FIGS. 4A and 4B are schematic diagrams illustrating a microfluidic detection device according to an embodiment of the present invention. FIG. 4A is a top view illustrating the structure of a microfluidic detection device according to an embodiment of the present invention. FIG. 4B is a side view illustrating the structure of a microfluidic detection device according to an embodiment of the present invention.

FIG. 5A to 5D are schematic diagrams illustrating a microfluidic detection device according to an embodiment of the present invention. FIGS. 5A and 5B are schematic diagrams showing the structure of branched microfluidic channel structure with or without sample application, respectively, and illustrating gates disposed between the reaction well and the detection well. FIG. 5C is a top view showing the detection wells of the branched microfluidic channel structure. FIG. 5D is a schematic diagram showing the reaction wells of the branched microfluidic channel structure for performing nucleic acid amplification.

### DETAILED DESCRIPTION

The following descriptions of the embodiments illustrate implementations of the present disclosure, and those skilled in the art of the present disclosure can readily understand the advantages and effects of the present disclosure in accordance with the contents herein. However, the embodiments of the present disclosure are not intended to limit the scope of the present disclosure. The present disclosure can be practiced or applied by other alternative embodiments, and every detail included in the present disclosure can be changed or modified in accordance with different aspects and applications without departing from the essentiality of the present disclosure.

The features such as a ratio, structure, and dimension shown in drawings accompanied with the present disclosure are simply used to cooperate with the contents disclosed herein for those skilled in the art to read and understand the present disclosure, rather than to limit the scope of implementation of the present disclosure. Thus, in the case that does not affect the purpose of the present disclosure and the effect brought by the present disclosure, any change in proportional relationships, structural modification, or dimensional adjustment should fall within the scope of the technical contents disclosed herein.

As used herein, “comprising” (and any variant or conjugation thereof, such as “comprise” or “comprises”), “including” (and any variant or conjugation thereof, such as “include” or “includes”), or “having” (and any variant or conjugation thereof, such as “have” or “has”) a specific element, unless otherwise specified, may include other elements such as components, ingredients, structures, regions, portions, devices, systems, steps, or connection relationships rather than exclude those elements.

The terms “on,” “upper,” “under,” “lower,” “deeper,” “front,” “between,” and “rear” described herein are simply used to clarify the embodiments of the present disclosure, rather than used to limit the scope of implementation of the present disclosure. Adjustments, interchanges, and alteration of relative positions and relationships thereof should be considered within the scope of implementation of the present disclosure if the technical contents of the present disclosure are not substantially changed.

The terms “first,” “second,” “third,” “fourth,” etc., used herein are simply used to describe or distinguish elements such as components, ingredients, structures, regions, portions, devices, or systems, rather than used to limit the scope of implementation of the present disclosure or to limit the spatial order of the elements. In addition, unless otherwise specified, the singular forms “a” and “the” used herein also include plural forms, and the terms “or” and “and/or” used herein are interchangeable.

The numeral ranges used herein are inclusive and combinable, and any numeral value that falls within the numeral scope herein can be taken as a maximum or minimum value to derive the sub-ranges therefrom. For example, it should be understood that the numeral range from “0.5 mm to 1.5 mm” comprises any sub-ranges between the minimum value of 0.5 mm and the maximum value of 1.5 mm, such as the sub-ranges from 1.0 mm to 1.5 mm, from 0.5 mm to 1.0 mm, and from 0.7 mm to 1.2 mm. Furthermore, any multiple numeral points used herein can be chosen as a maximum or minimum value to derive the numeral ranges therefrom. For example, 0.7 mm, 0.9 mm, and 1.4 mm can derive the numeral ranges of 0.5 mm to 1.0 mm, 0.7 mm to 1.2 mm, or 1.0 mm to 1.5 mm.

The terms “settling well” and “detection well” used herein are interchangeable, unless otherwise specified.

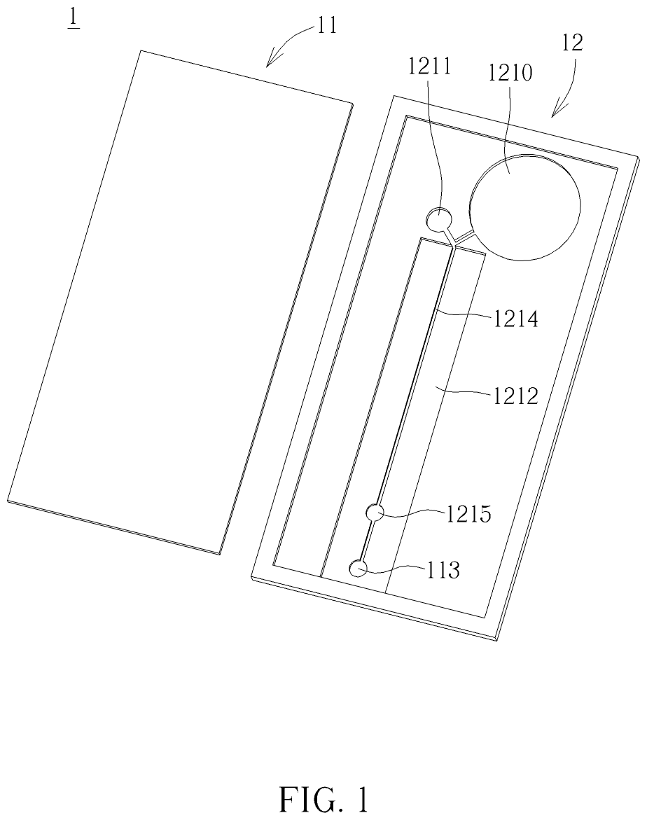

FIG. 1 shows the microfluidic detection device 1 according to at least one embodiment of the present disclosure, including a dynamic well 1210, a sample well 1211, a channel 1214, a reaction well 1212, a settling well 1215, and an air hole 113. It should be noted that the quantity and connection of each of the parts are exemplary and can be increased, decreased, or altered according to actual needs. As shown in FIG. 1, the dynamic well 1210 is used for facilitating the flow of the sample fluid through the microfluidic detection device 1 via exerting a gas pressure which moves the sample fluid through the sample well 1211 and into the channel 1214; sample fluid is dropped into the sample well 1211 and flows to the afterward sections through the channel 1214; the reaction well 1212 contains color markers, e.g. antibody-colloidal gold conjugates, coated directly on the reaction well 1212 or on a test paper disposed on the reaction well 1212, and the color markers are mixed with and binding to the target molecules in the sample fluid; the settling well 1215 may be deeper or wider than channel 1214 to realize that the flow rate is inversely proportional to the area to achieve velocity reduction; and the air hole 113 is designed to prevent the air bubbles in the sample from blocking the microfluidic channel structure 121. In the embodiment, the settling well 1215 may be deeper than the channel 1214 enabling well mixing and binding of the color markers and target molecules in the sample fluid. In the embodiment, the diameter of the dynamic well 1210 may be 13 mm, and the depth of the dynamic well 1210 may be 0.5 mm. In the embodiment, the diameter of the sample well 1211 may be 3 mm, and the depth of the sample well 1211 may be 0.5 mm. In the embodiment, the width of the channel 1214 may be 0.4 mm, and the depth of the channel 1214 may be 0.5 mm. In the embodiment, the length of the reaction well 1212 may be 42 mm, and the width of the reaction well 1212 may be 0.5 mm. In the embodiment, the diameter of the settling well 1215 may be 2 mm, and the depth of the settling well 1215 may be 0.5 mm. In the embodiment, the length of the microfluidic detection device 1 may be 60 mm, the width of the microfluidic detection device 1 may be 30 mm, and the height of the microfluidic detection device 1 may be 1.8 mm. In the embodiment, the length of the base 12 may be 56 mm, the width of the base 12 may be 26 mm, and the height of the base 12 may be 0.8 mm. In the embodiment, the length of the lid 11 may be 56 mm, the width of the lid 11 may be 26 mm, and the height of the lid 11 may be 10 mm. In the embodiment, the diameter of the air hole 113 is 3 mm.

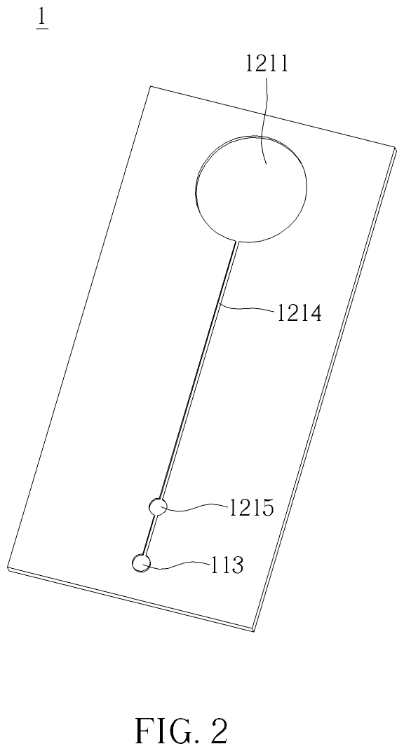

FIG. 2 shows the microfluidic detection device 1 according to at least one embodiment of the present disclosure, including a sample well 1211, a channel 1214, a settling well 1215, and an air hole 113. It should be noted that the quantity and connection of each of the parts are exemplary and can be increased, decreased, or altered according to actual needs. As shown in FIG. 2, sample fluid is dropped into the sample well 1211 and flows to the afterward sections through the channel 1214; the settling well 1215 may be deeper or wider than channel 1214 to realize that the flow rate is inversely proportional to the area to achieve velocity reduction; and the air hole 113 is designed to prevent the air bubbles in the sample from blocking the microfluidic channel structure 121. In the embodiment, the diameter of the sample well 1211 may be 12 mm, and the depth of the sample well 1211 may be 0.5 mm. In the embodiment, the width of the channel 1214 may be 0.35 mm, and the depth of the channel 1214 may be 0.5 mm. In the embodiment, the diameter of the settling well 1215 may be 1.8 mm, and the depth of the settling well 1215 may be 0.5 mm. In the embodiment, the length of the microfluidic detection device 1 may be 60 mm, the width of the microfluidic detection device 1 may be 30 mm, and the height of the microfluidic detection device 1 may be 1.8 mm. In the embodiment, the diameter of the air hole 113 is 3 mm.

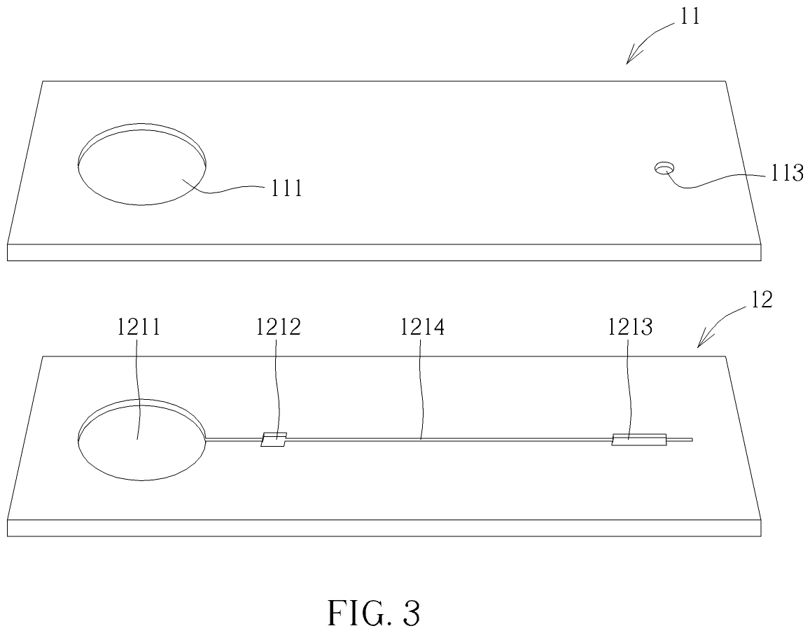

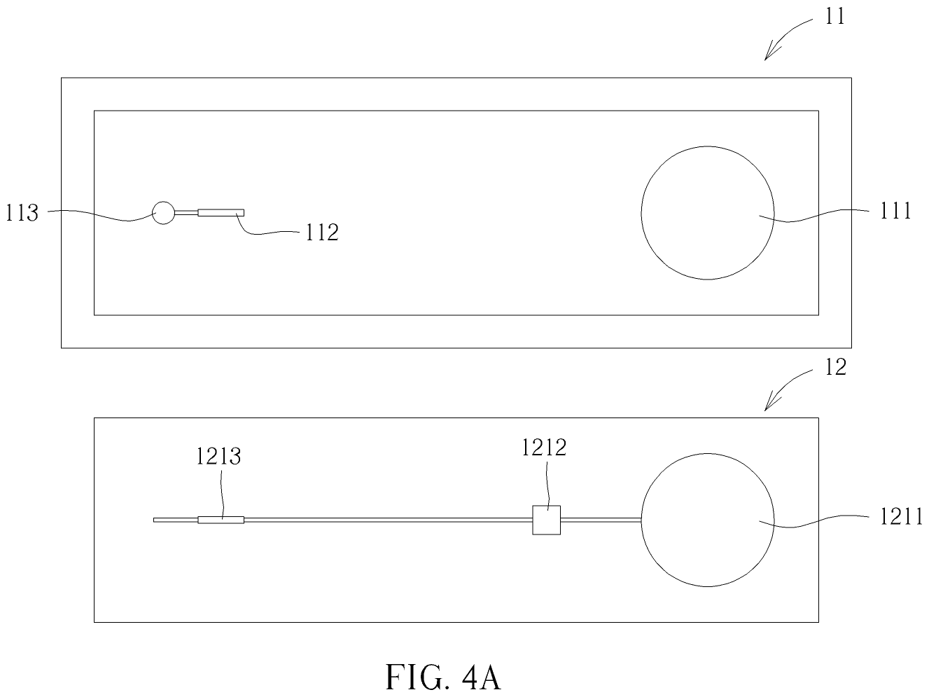

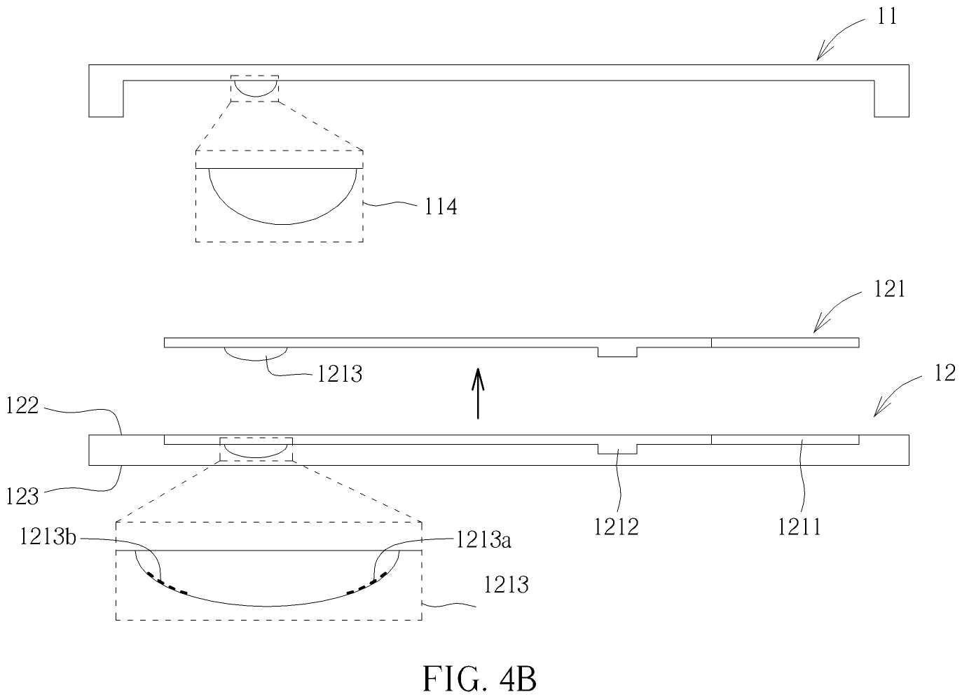

FIGS. 3, 4A and 4B show the microfluidic detection device 1 according to at least one embodiment of the present disclosure, including a base 12 and a lid 11. In the embodiment, the base 12 comprises a microfluidic channel structure 121 on the surface and the microfluidic channel structure 121 comprises a sample well 1211, a reaction well 1212, a detection well 1213, and a channel 1214 connecting the sample well 1211, the reaction well 1212, and the detection well 1213. In the embodiment, the detection well 1213 has a control region 1213a and a test region 1213b, and the detection well 1213 is a recess deeper than the channel 1213, wherein the recess is along the direction from an upper surface 122 of the base 12 to a lower surface 123 of the base 1. In the embodiment, the lid 11 has a sample-through hole 111 corresponding to the sample well 1211 of the base 12, a detection window 112 corresponding to the detection well 1213 of the base 12, for observation of the color development of the control region 1213a and the test region 1213b, and a protrusion 114 corresponding to the recess (i.e. the detection well 1213) of the base 12 to form a space between the protrusion and the recess (FIGS. 4A and 4B). It should be noted that the quantity and connection of each of the parts are exemplary and can be increased, decreased, or altered according to actual needs. In the embodiment, the diameter of the sample well 1211 may be 10 mm, and the depth of the sample well 1211 may be 0.5 mm. In the embodiment, the length of the channel 1214 may be 37 mm, and the depth of the channel 1214 may be 0.5 mm. In the embodiment, the length of the reaction well 1212 may be 5 mm, the width of the reaction well 1212 may be 5 mm, and the depth of the reaction well 121 may be 1 mm. In the embodiment, the length of the detection well 1213 may be 3 mm, the width of the detection well 1213 may be 0.35 mm, and the depth of the settling well 1215 may be 0.5 mm. In the embodiment, the length of the base 12 may be 55 mm, the width of the base 12 may be 15 mm, and the height of the base 12 may be 1.8 mm. In the embodiment, the length of the lid 11 may be 60 mm, the width of the lid 11 may be 20 mm, and the height of the lid 11 may be 2.5 mm. In the embodiment, the diameter of the air hole 113 is 1.5 mm. In the embodiment, the x-y-z value of the protrusion 114 is 0.35-3.131-0.752 mm. In the embodiment, the x-y-z value of the recess 114 is 0.35-4-1.087 mm.

As shown in FIGS. 3, 4A and 4B, during the test, sample fluid is dropped into the sample well 1211, and the sample fluid flows to the afterward sections through the channel 1214. The sample fluid firstly flows to the reaction well 1212, containing color markers, e.g. antibody-colloidal gold conjugates, coated directly on the reaction well 1212 or on a test paper disposed on the reaction well 1212, and the color markers are mixed with and binding to the target molecules in the sample fluid. For good mixing and binding, the reaction well 1212 may be deeper than the channel 1214 along the direction from the upper surface 122 of the base 12 to the lower surface 123 of the base 12. Then, the sample fluid flows to the detection well 1213. The recess of the base 12 and the protrusion 114 of the lid 11 forms a curved space here to reduce the rate of the sample flow (FIGS. 4A and 4B). In the embodiment, the recess and the protrusion 114 are arc-shaped and the curved space is therefore a curved channel, U-shaped tube (FIGS. 4A and 4B). The reduced rate of sample flow allows more time for the target molecules (bound to the color marker) in the sample to react with or captured by the antibody coating on the control region 1213a and test region 1213b, thereby improving the color development. In the embodiment, the detection well 1213 contains a signal booster. The signal booster may be added to or coated on the curved space. Hence, the expression of the antibody-antigen signal in the test region 1213b can be enhanced, strengthening the sensitivity of the microfluidic detection device 1.

As shown in FIGS. 3 and 4A, at least one air hole 113 is designed to prevent the air bubbles in the sample from blocking the microfluidic channel structure 121. The air hole 113 may be provided on the lid 11 and the locations of which correspond to the microfluidic channel structure 121, e.g. behind the detection well.

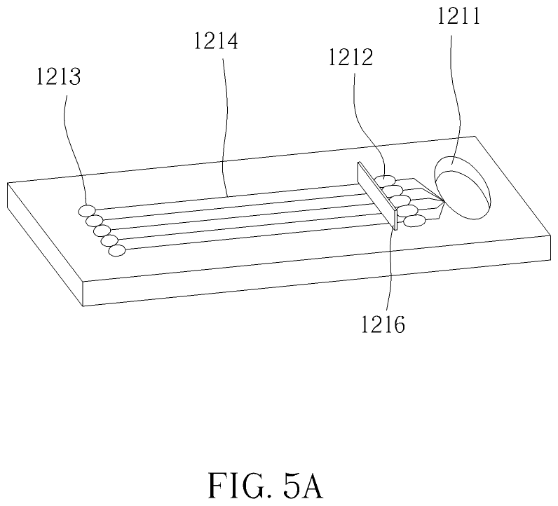

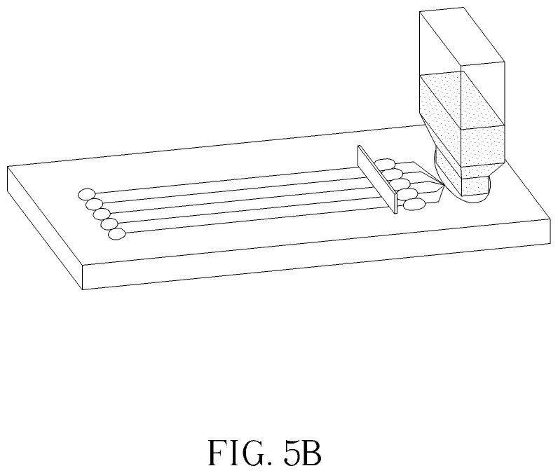

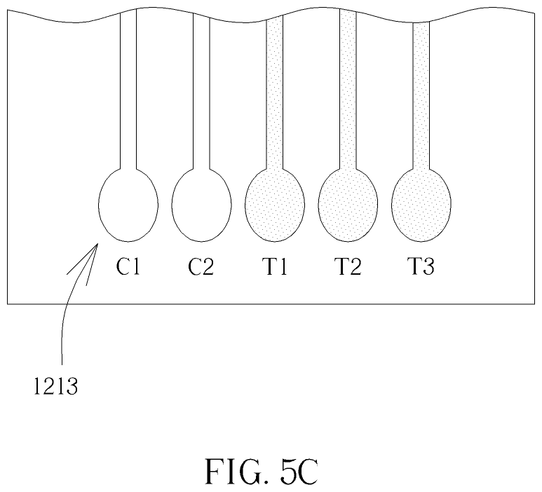

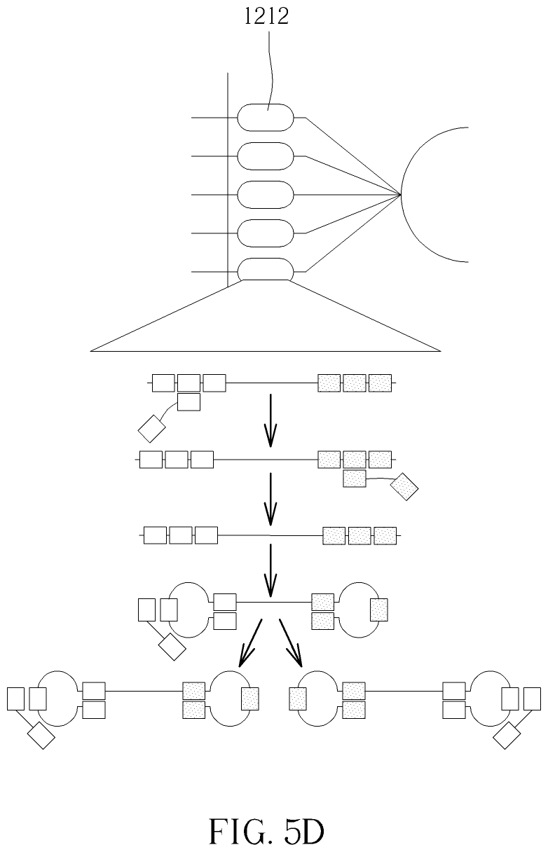

FIG. 5A to 5D show the microfluidic detection device 1 according to at least one embodiment of the present disclosure, including a branched microfluidic channel structure 121 to detect various target molecules simultaneously. In the embodiment, the channel 1214 between the sample well 1211 and the reaction well 1212 is divided into 2 or more branches, and each branch comprises the reaction well 1212 and the detection well 1213, respectively. The structure of the reaction wells 1212 and the detection wells 1213 in each branch may be the same as mentioned above, except that the color marker contained in the reaction well 1212 and the antibody coated on the detection well 1213 are directed against different target molecules. As shown in FIGS. 5A and 5B, sample fluid is dropped into the sample well 1211, and the sample fluid flows to the afterward sections through each channel 1214 of different branches. As shown in FIG. 5B, the sample liquid was subjected to nucleic acid proliferation in reaction well 1212 for 20 min, after which gate 1216 was raised. Gate 1216 is no lower than the channel 1214 in height and is wide enough to cover all branches, and extraction may be employed to remove the valve and allow the sample fluid to pass through. As shown in FIG. 5B, the sample fluid may remain in the reaction wells 1212 for performing nucleic acid amplification for 20 min, and then the gates 1216 are lifted allowing the sample fluid to pass through. As shown in FIG. 5C, the C region (including C1, C2, etc.) can be regarded as, but not necessarily, the control group, and generally includes the positive control group and the negative control group, with the positive control group showing a certain response and the negative control group showing a certain non-response, whereas the T region (including T1, T2, T3, etc.) is the target molecule to be detected. As shown in FIG. 5D, the reaction wells 1212 of the branched microfluidic channel structure 121 are used for performing nucleic acid amplification.

### EXAMPLES

### Example 1: The Adhesion Test of the Lid and Base of the Microfluidic Detection Device

In order to test the ability to fix the lid and base by acetone with modified/unmodified lid and base, the lid and base are coated with or without hydrophilic membrane. The steps for adhesion test of modified and unmodified microfluidic detection device of the present disclosure are as follows:
1. Fix the lid and the base by applying acetone around the microfluidic channel structure with a medicine spoon or a pipette tip.
2. Drop 100 μl dye solution in the sample well to test the adhesive ability (see Table 1).

TABLE 1

| | Tools | Results |
|---|---|---|
| Unmodified lid and base | medicine spoon | mild leakage |
| | tip | severe leakage |
| Modified lid and base | tip | severe leakage |

### Example 2: The Microfluidic Detection Device for HPV Assay

The HPV test kit contains a manual, a package insert, a urine cup, a sample buffer tube, a microfluidic detection device, and a dropper. The steps for performing the RDT in order to detect the pathogen by the microfluidic detection device of the present disclosure are as follows:
1. Take 100 cc of midstream urine, take 100 μl as a sample using the dropper, and mix it thoroughly with the sample buffer to obtain a sample mixture.
2. Drop the sample mixture into a sample well on the microfluidic detection device, and the sample mixture starts to move automatically through capillary action after entering a channel.
3. While the sample mixture flowed through a reaction well containing a test paper coated with conjugated nano-gold HPV16 L1 (The L1 Major Capsid Protein of Human Papillomavirus Type 16) antibodies, the potential antigens HPV16 L1 inside the sample mixture bound to the conjugated nano-gold HPV16 L1 antibodies.
4. After that, the sample mixture entered a detection well and reached a control region, and antibodies contained in the control region reacted with the “unbound” conjugated nano-gold HPV16 L1 antibodies. Unbound antibodies were thus captured, deposited, aggregated, and the control region was colored, i.e., the test result is valid. Otherwise, if the control region was not colored, the test result was considered invalid.
5. The sample mixture continued to move into a curved channel between the control region and a test region described later. The flow rate was reduced and the reaction time between the sample mixture and antibodies was increased. Also, the impurities and unwanted substances were deposited at the bottom of the curved channel to reduce interference during the test.
6. Finally, the sample mixture reached the test region containing the other HPV16 L1 antibodies. The “binding” conjugated nano-gold HPV16 L1 antibodies reacted with the other HPV16 L1 antibodies and the binding antibodies were thus captured, deposited, and aggregated. The test result was positive if the test region was colored, otherwise the test result is negative.

### Example 3: Examination of Flow Velocity Measurement with Modified/Unmodified Microfluidic Detection Device

In order to test the flow velocity of the modified/unmodified microfluidic detection device, the time taken for the sample fluid to flow through the microfluidic channel structure is measured. The steps for flow velocity measurement of modified and unmodified microfluidic detection device of the present disclosure are as follows:
1. The modified lid and base are activated by UV light exposure for 30 min, since the hydrophilic membrane coating is inactivated at room temperature.
2. Drop 1004 dye solution in the sample well and the time taken for the sample fluid to flow through the microfluidic channel structure is measured (see Table 2).

TABLE 2

| | | Artificial |
|---|---|---|
| Water | | Urea |
| base + lid | base | base + lid |

| | fixed | not fixed | N/A | not fixed |
|---|---|---|---|---|
| Unmodified | >20 min | not | — | not |
| microfluidic | | moveable | | moveable |
| detection | | | | |
| device | | | | |
| Modified | — | 45 s | 45 s | 27 s |
| microfluidic | | (remains | (remains | |
| detection | | in the | in the | |
| device | | recess) | recess) | |
| (inactivated) | | | | |
| Modified | — | 15 s | 27 s | 15 s |
| microfluidic | | | | |
| detection | | | | |
| device | | | | |
| (activated) | | | | |

Accordingly, the flow velocity of the ionic solution is faster than the flow velocity of the water.

In conclusion, one of purposes of the present disclosure is to provide a faster and more effective way for cancer screening and early detection for early treatment. To improve usability and convenience, body fluid from the subject (e.g., peripheral blood, sera, plasma, ascites, urine, cerebrospinal fluid (CSF), sputum, saliva, bone marrow, synovial fluid, aqueous humor, bronchoalveolar lavage fluid, semen, prostatic fluid, Cowper's fluid or pre-ejaculatory fluid, sweat, fecal matter, tears, cyst fluid, pleural and peritoneal fluid, pericardial fluid, lymph, chyme, chyle, bile, interstitial fluid, pus, sebum, vomit, mucosal secretion, stool water, pancreatic juice, lavage fluids from sinus cavities, bronchopulmonary aspirates or other lavage fluids) can be used as a sample for the microfluidic detection device of the present application. For instance, some embodiments of the present application develop a urine-based HPV RDT for cervical cancer, some other embodiments provide a saliva-based HPV RDT for head and neck cancer. These embodiments can all achieve high-efficiency and convenience in the cancer screening and early detection. The microfluidic detection device of the present disclosure can also assist in a long-term follow-up treatment.

Furthermore, compared with the traditional test paper RDTs, the microfluidic detection device of the present disclosure can reduce the demand for sample volume, enhance the signal of the antigen-antibody reaction through the specific microfluidic structure (or optionally, the signal booster), and prevent the clogging of the flow channel caused by the ingredients of the sample. Hence, the present disclosure can enhance the specificity and color development of RDTs and thereby expand the range of diseases for which RDTs are applicable, especially cervical cancer and head and neck cancer, but the present disclosure is not limited thereto.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A microfluidic detection device, comprising:
a base with a microfluidic channel structure formed on the base, wherein the microfluidic channel structure comprises:
a sample well for loading a sample,
a detection well has a first reagent for reacting with the sample, and
a channel connecting the sample well and the detection well,
wherein the detection well has a recess deeper than the channel; and
a lid covering the base, having:
a protrusion corresponding to the recess to form a space between the protrusion and the recess.

2. The microfluidic detection device of claim 1, wherein the recess is along the direction from an upper surface of the base to a lower surface of the base.

3. The microfluidic detection device of claim 1, wherein the recess and the protrusion are arc-shaped.

4. The microfluidic detection device of claim 3, wherein the space between the recess and the protrusion forms a curved channel.

5. The microfluidic detection device of claim 1, wherein the first reagent comprises a signal booster.

6. The microfluidic detection device of claim 1, wherein the lid further has at least one air hole corresponding to the microfluidic channel structure.

7. The microfluidic detection device of claim 1, wherein the detection well has a control region and a test region.

8. The microfluidic detection device of claim 1, wherein the lid further has:
a sample through hole corresponding to the sample well; and
a detection window corresponding to the detection well.

9. The microfluidic detection device of claim 1, wherein the microfluidic channel structure further comprises a reaction well between the sample well and the detection well, the reaction well has a second reagent for reacting with the sample, and wherein the sample well, the reacting well, and the detection well are connected by the channel.

10. The microfluidic detection device of claim 9, wherein the reaction well is deeper than the channel along the direction from the upper surface of the base to the lower surface of the base.

11. The microfluidic detection device of claim 9, wherein the microfluidic channel structure is branched, and each branch of the microfluidic channel structure comprises the reaction well and the detection well.

12. The microfluidic detection device of claim 11, further comprises a gate disposed between the reaction well and the detection well to control the flow of fluid from the reaction well to the detection well.

13. The microfluidic detection device of claim 9, wherein the second reagent comprises antibody-colloidal gold conjugates.

14. The microfluidic detection device of claim 13, wherein the antibody-colloidal gold conjugates are coated on a test paper disposed on the reaction well.

15. The microfluidic detection device of claim 9, wherein the second reagent comprises a reagent for nucleic acid amplification.

16. The microfluidic detection device of claim 15, wherein the nucleic acid amplification is selected from the group consisting of nucleic acid sequence-based amplification (NASBA), recombinase polymerase amplification (RPA), loop-mediated isothermal amplification (LAMP), strand displacement amplification (SDA), helicase-dependent amplification (HDA), rolling circle amplification (RCA), and whole genome amplification (WGA).

17. A method for rapid diagnostic testing, comprising:
obtaining a sample from a subject in need thereof;
loading the sample to the microfluidic detection device of claim 1; and
detecting a biochemical value or a disease pathogen by the microfluidic detection device.

18. The method of claim 17, wherein the disease pathogen is selected from the group consisting of bacteria, viruses, fungi, protists, and parasitic worms.
